(19) United States
(12) Patent Application Publication
(10) Pub. No.: US 2024/0312582 A1
(43) Pub. Date: Sep. 19, 2024

(54) INCIDENT REPORT AUDITING SYSTEMS AND METHODS

(71) Applicant: Revelar Technologies LLC, Forest Hills, NY (US)

(72) Inventors: Gabriel Mayer, Forest Hills, NY (US); Beau Jensen Waiker, Irvine, CA (US); Joe Matthew Queen, Rome, GA (US); Linda Yun Kim, Irvine, CA (US)

(21) Appl. No.: 18/182,983

(22) Filed: Mar. 13, 2023

(51) Int. Cl. G16H 15/00; G16H 40/20

(52) U.S. Cl. CPC G16H 15/00; G16H 40/20

(57) ABSTRACT

Systems and methods of the inventive subject matter are directed to auditing actions that take place in a facility, such as a care facility, where actions are undertaken by people and where those actions and associated documentation can benefit from auditing. In one example, incidents that take place in a care facility can be documented and reported, and embodiments of the inventive subject matter can then be used to audit the incident reports to, e.g., minimize liability and maximize quality of care.

### FIELD OF THE INVENTION

The field of the invention is incident report auditing.

### BACKGROUND

The background description includes information that may be useful in understanding the present invention. It is not an admission that any of the information provided in this application is prior art or relevant to the presently claimed invention, or that any publication specifically or implicitly referenced is prior art.

Consistent, accurate record keeping in any context can help to eliminate liability exposure. In medicine and health care, for example, record keeping is a critical aspect of providing care, and incomplete medical records can lead to unnecessary liability exposure. Incomplete records can create legal defense challenges that may ultimately result in higher insurance premiums.

In the context of care facilities (e.g., clinics, hospitals, nursing facilities, elder care facilities and so on), directors of nursing (DONs) must audit electronic medical records (EMRs) every day. Auditing is a highly manual process that can be time consuming and tedious to perform properly. As a result, auditing is not always performed perfectly or with perfect regularity.

Many care facilities deliver high quality care, but gaps in record keeping can give rise to many issues in, e.g., situations where lawsuits are filed, insurance claims are submitted, cases are litigated, etc. It has been found in some studies that 70-80% of all skilled nursing general and professional liability claims have documentation deficiencies and that 72% of all electronic health record (EHR) related health issues are because of documentation deficiencies.

To minimize exposure to liability traces back to improved record keeping, and one way to improve record keeping is to improve the auditing process. Audits are intended to catch gaps in, e.g., contemporaneously kept records, and if these gaps are caught early, they can accurately be fixed. By ensuring accurate and consistent record keeping, care facilities can reduce liability exposure, which in turn can result in reduced insurance premiums.

A need therefore exists for tools that facilitate the process of auditing medical records. These tools should make it easier to review and modify records, automate as much of the auditing process as possible, and as a result, reduce exposure to liability.

### SUMMARY OF THE INVENTION

The present invention provides systems and methods directed to facilitating the auditing of care facilities' incident reports and activities to improve care and to reduce liability. A first aspect of the inventive subject matter is directed to a method of auditing an incident report generated at a care facility, the method comprising the steps of: receiving, by a platform server, the incident report from the care facility, the incident report comprising documentation of the incident; applying, by the platform server, tags to the documentation, where each tag has an associated tag status; receiving, by the platform server, an audit selection, the audit selection comprising the incident report; displaying the documentation with the applied tags and tag statuses; receiving, by the platform server, changes to the documentation, the applied tags, and the tag statuses; and displaying a summary of all changes made to the documentation, the applied tags, and the tag statuses.

In some embodiments, the method also includes the step of identifying, by the platform server, the incident report as having been audited and storing a time of audit completion. The method can also include the step of receiving, after displaying the summary, an indication of whether additional documentation is missing, and in some embodiments, upon a determination that the additional documentation is missing, the platform server sends audit information to the care facility for additional review. Each tag status can include at least one of a “missing” status and an “incomplete” status.

Another aspect of the inventive subject matter is directed to a method of auditing incident reports generated at care facilities, the method comprising the steps of: receiving, by a platform server, an incident report generated at a care facility, the incident report comprising documentation of an incident; applying, by the platform server, a documentation tag to a portion of the documentation, where the documentation tag has a tag status; storing, by the platform server, statistically useful information relating to the portion of the documentation based on the tag status; receiving, by the platform server, an audit selection, the audit selection comprising the incident report; displaying the documentation, including the portion of the documentation having the documentation tag and the tag status; receiving, by the platform server, changes to documentation and tags; and displaying a summary of changes made to documentation, including changes to the documentation tag and the tag status.

In some embodiments, the tag status includes at least one of a “missing” status and an “incomplete” status. The method can also include the step of identifying, by the platform server, the incident report as having been audited and storing a time of audit completion. In some embodiments, the method also includes the step of receiving, after displaying the summary, an indication of whether additional documentation is missing. Upon a determination that the additional documentation is missing, the platform server can then send audit information to the care facility for additional review. In some embodiments, the statistically useful information can be used in a statistical analysis.

One should appreciate that the disclosed subject matter provides many advantageous technical effects including improved auditing and reviewing capabilities for care facilities that can reduce liability risks while simultaneously improving quality of care.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a flow chart describing steps of an embodiment of the inventive subject matter.

FIG. 2 is a user interface screen showing a listing of incident reports that can be audited.

FIG. 3 is a user interface that shows documentation associated with a selected incident report.

FIG. 4 is a user interface that shows injuries associated with the selected incident report.

FIG. 5 is a user interface that shows documentation that has been flagged as missing in the selected incident report.

FIG. 6 is a user interface that shows actions that have been taken according to the selected incident report.

FIG. 7 is a user interface showing comments relating to aspects of the selected incident report.

FIG. 8 is a user interface showing care plans that are associated with the selected incident report.

FIG. 9 is a user interface showing a summary of all changes made throughout auditing the selected incident report.

FIG. 10 shows schematically how computing devices and the platform server communicate with each other in embodiments of the inventive subject matter.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used in the description in this application and throughout the claims that follow, the meaning of “a,” “an,” and “the” includes plural reference unless the context clearly dictates otherwise. Also, as used in the description in this application, the meaning of “in” includes “in” and “on” unless the context clearly dictates otherwise.

Also, as used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth in this application should be interpreted as being inclusive of their endpoints and open-ended ranges should be interpreted to include only commercially practical values. Similarly, all lists of values should be considered as inclusive of intermediate values unless the context indicates the contrary.

It should be noted that any language directed to a computer should be read to include any suitable combination of computing devices, including servers, interfaces, systems, databases, agents, peers, Engines, controllers, or other types of computing devices operating individually or collectively. One should appreciate the computing devices comprise a processor configured to execute software instructions stored on a tangible, non- transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network. The following description includes information that may be useful in understanding the present invention. It is not an admission that any of the information provided in this application is prior art or relevant to the presently claimed invention, or that any publication specifically or implicitly referenced is prior art.

Systems of methods of the inventive subject matter are directed to incident report auditing for facilities such as care facilities. In, e.g., an elder care facility, incidents regularly occur that result in incident reports that can be useful to keep track of. In some instances, incident reports can be required by law. Embodiments are designed to improve the quality of incident reports and to generate statistics related to incident reports that can together be used to minimize liability, minimize future incidents, improve quality of care, and so on. Embodiments can be implemented as software as a service that exists on a platform server and can be accessed by computing devices such as computers, cell phones, tablets, and so on via application, software as a service, web browser, etc. When, for example, the platform server is described as taking an action such as “displaying” some content, this is intended to be interpreted as the platform server causing a computing device to display some content, either locally or remotely. This comports with ordinary understanding of computing, generally, and enables simpler discussion of the inventive subject matter.

Systems and methods of the inventive subject matter can be used for several purposes, including lowering institutional risk, improving underwriting accuracy, and lower cost of insurance claims. Institutional risk refers to an institution's overall risk of giving rise to insurance claims or other matters that implicate an institution's insurance policy. Institutional risk can be lowered by helping employees to show that required standards of care are upheld throughout operations. These risks can also be reduced by ensuring staff is taking proper steps to provide high-quality care, by allowing for fast, standardized communications, by facilitating creating of consistent routines, and by helping analysts identify patterns that can inform changes and mitigation plans that can improve operations.

Systems and methods of the inventive subject matter can improve underwriting accuracy by, e.g., introducing new stats or variables for underwriters to track. Underwriters can be made aware of: a number of incident reports that are missing documentation; a rate of new incidents; a breakdown of incident types and severities; factors that contribute to the probability of a lawsuit or insurance claim; and trendlines for changes in risk over time.

Systems and methods of the inventive subject matter can lower claim costs by, e.g., offering more information relating to a claim that can be used to better determine how a claim can be resolved and by reducing administrative work during phases of a claim such as pre-litigation preparation as well as litigation related processes such as discovery. Discussion of the inventive subject matter in this application is directed to systems and methods that can be used by, e.g., care facilities for the purposes described above.

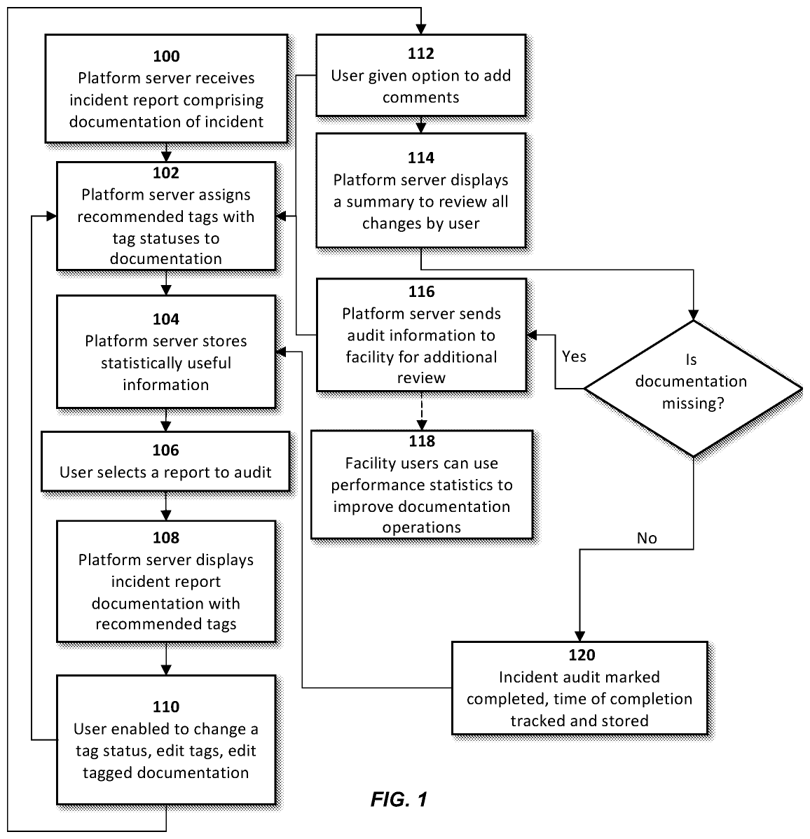

FIG. 1 is a flowchart showing how a platform server can be interacted with by users to give rise to an incident auditing system of the inventive subject matter. In step 100, the platform server gathers documentation for an incident report. Documentation can be gathered by, e.g., nurses working in a hospital or other care facility inputting incident reports. In this application, an incident is, e.g., a fall, an injury, a medical event, or other noteworthy incident, and a report of that incident comprises documentation describing that incident. For example, a care specialist such as a nurse may report a patient falling out of bed. In that case, the report includes documentation of that fall, such as whether the patient was injured, whether anyone was notified, what time the fall occurred, and so on.

Thus, if a patient experiences a reportable incident (e.g., a medical event, a fall, or any other noteworthy incident), information about that incident can be gathered by one or more nurses and submitted as an incident report with associated documentation. In some embodiments, reports that are available for submission are based on compliance needs. For example, an insurance carrier may require certain incidents to be reported while not requiring others. A fall may be important to generate a report for, while a stubbed toe may not, and whether to generate a report can be influenced by compliance needs. Incidents that can be reported can additionally be set on an as-needed basis, depending on the specific standards or requirements for a particular entity using the system.

In step 102, the platform server, having received an incident report and associated documentation, then applies tags to the documentation, where each tag can have an associated tag status. This process is automated by the platform server and is based on content and completeness of documentation received by the platform server. Documentation received by the platform server can vary by incident report type and by the humans that handle generating that documentation, and thus documentation tags and associated documentation tag statuses can be based on, e.g., what information is received, how much information is received, whether additional information is needed, and so on.

A variety of different documentation tags can be used, and those tags can indicate different documentation statuses including that some aspect of the documentation is completed, missing (e.g., flagged as missing some information in existing documentation or missing documentation entirely), should not be flagged, requires confirmation, and so on. When some aspect of documentation associated with an incident report is tagged as “do not flag,” for example, the tag indicates that a specific report had no need for that aspect of documentation. In some embodiments, this tag can only be updated by, e.g., a system manager such as a director of nursing or other party responsible for auditing incident reports. When some aspect of an incident report's documentation is tagged as needing confirmation, that indicates that aspect of the documentation should be confirmed by an auditor. As used in this application, the term “user” refers to any user of the platform, which can include a person acting as an auditor. All tags can be human reviewed (e.g., by an auditor), and human review can help to ensure tags are accurate.

In step 104, the platform server stores information that can be statistically useful, and that information can be stored based on documentation statuses for use in statistical analyses. For example, if a fall has been reported and some portion of documentation associated with that fall is tagged as “missing,” the platform server can first store that a fall incident type occurred along with the date and timestamp, and it can then identify what information is tagged as missing. Once the missing information has been identified, it can be corrected by a user, and the time it takes for each “missing” tag to be fixed as well as the time it takes for the entire report to be audited is recorded and stored. Other tag statuses include “incomplete,” “complete,” “needs review,” and “in progress.” In some embodiments, documentation metadata can be stored based on a tag status, such as a time the documentation was generated, a time an incident occurred, an incident category, and so on.

The platform server can also, in this step, store performance statistics. Performance in this context refers to the performance of a care facility or group overall as it relates to different reports. For example, performance statistics can include: times when incidents occur (e.g., shift, afternoon shift, night shift); a number of flags per audit stage (e.g., details, injuries, actions, notes, care plans); a number of incidents that occur over a period of time; a number of incidents per incident category; a number of completed review audits over a period of time; and time taken to complete audits of various reports (e.g., measured in days, hours, or the like). In one example, a nursing facility may be informed by a performance statistic that its patients experienced six falls where three were flagged for injury documentation and one was flagged for action (such as notifying family).

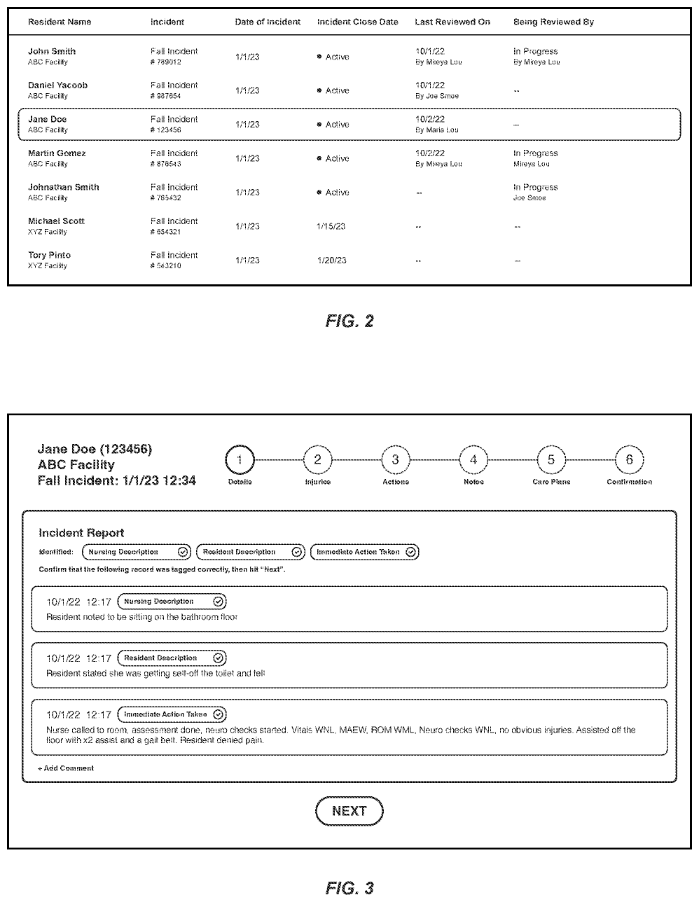

In step 106, a user selects a report to audit. Users are presented with a list of different reports (as shown in, e.g., FIG. 2). From there, the platform server displays documentation of the selected incident report per step 108. In this step, recommended actions can also be shown. The platform server generates recommended actions based on, e.g., documentation tags and report type as well as the content of the associated documentation of that report. For example, if a fall is reported, the platform server can recommend that a pain evaluation take place (as shown in FIG. 5), or if family notification is required for an injury report, then the platform server can recommend family notification is one is not documented in the incident report.

Steps 108 through 112 are related to an audit flow, and step 114 is the final step to complete an audit. Once a user has selected a report to audit and the platform server has displayed recommendations, the user can then go through an audit flow one or more times, which includes the ability to change tag statuses or edit tags (e.g., add, remove, or change) per step 110, the ability to add comments per step 112, and being presented with a summary of all changes that occur during the audit per step 114. Throughout FIGS. 3-9, all or some of steps 110, 112, and 114 can be undertaken depending on circumstances, details, and statuses of the documentation associated with an incident report. In other words, each of the steps in an audit flow can occur multiple times for different portions of documentation presented by a user interface.

In an audit flow, a user is presented with an option to revise tagged documentation statuses per step 110. Documentation tags and statuses can be added in part by the platform server in step 108, which can reduce administrative burden. For example, if while auditing a fall report a user may see that the “injury” portion of documentation is tagged as “missing,” that user may add an injury description if one exists. By doing so, the “missing” tag status would no longer be applicable, and the user could, per step 110, revise that tag status or edit tags. An arrow connecting this step to step 102 is meant to indicate that step 110 provides feedback to the platform server when a tag status is changed or a tag is edited by the user. For example, each time a user reviews an aspect of documentation that has been tagged, that user can be prompted to revise the associated tag status or edit the tag before continuing to review documentation and associated tags and tag statuses.

Per step 112, users can be prompted to add comments. This is a step that gives users an opportunity to provide additional context to an incident report. Comments also can be about the incident reports themselves (e.g., a note to nurses about how best to prepare a certain type of incident report). Like step 110, step 112 is linked back to step 102 because comments made during this step can update an incident report which could require the platform server to update tags or tag statuses.

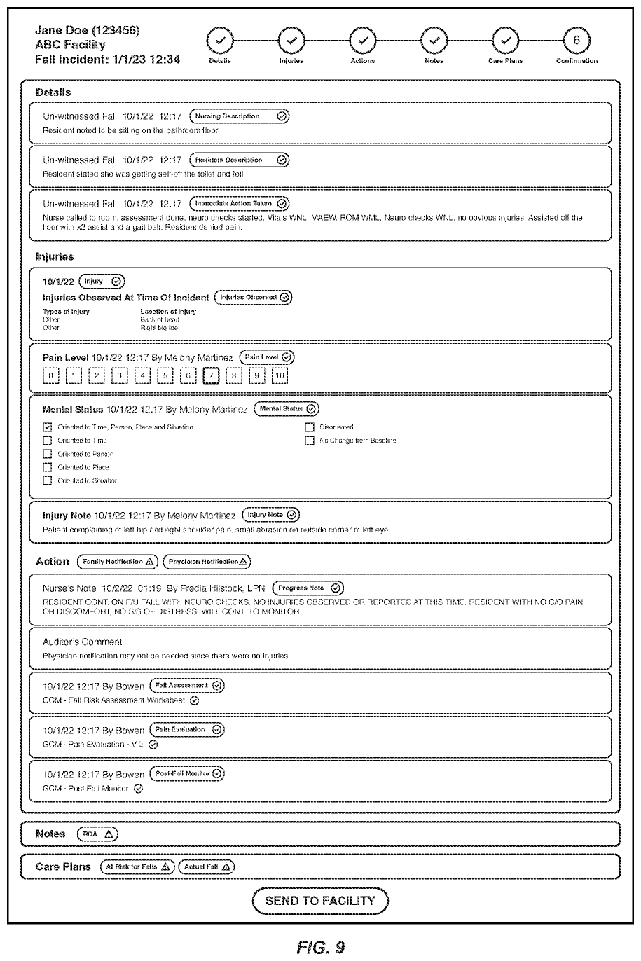

Once a review has been completed with all changes entered, the platform server provides a summary of those changes for the user to review, per step 114. This is also shown in FIG. 9. This summary gives users a way to ensure accuracy and completeness of changes. If a user sees an issue, they can make corrections in this step. Thus, when a user reviews all changes, they must check to see if any documentation or portion of documentation remains missing. If documentation is missing, the platform server in step 116 provides the incident report to a facility so that facility (e.g., the people working at a facility such as managers, directors, or anyone else responsible for the task of reviewing and fixing incident documentation after an audit has occurred) can fix and review the incident documentation, e.g., based on identified missing documentation. This step, again, links back to step 102 as it can result in changes to documentation tags, tag statuses, and the like. Once step 116 is completed, facility users can use statistics associated with the entire auditing and review process to improve documentation operations per step 118. Statistically useful information stored in, e.g., step 104 can be used in statistical analyses that can then be used by a care facility to improve or optimize its documentation operations, depending on its desired goal (e.g., to lower insurance premiums, reduce risk, and so on).

Steps 108, 110, and 112 can be repeated as many times as needed before moving to step 114, depending on user interface requirements. In some embodiments, steps 108 through 112 can be repeated on different screens of a user interface as many times as needed to go through an entire incident report before moving on to step 114. Using multiple screens to show documentation from an incident report is how the user interface in FIGS. 2-9, below, gets through auditing all portions of an example incident report.

If no documentation is missing, then, in step 120, the incident audit is marked as completed and stats associated with that audit are tracked and stored. Some stats include time of audit completion, time taken to complete audit, number of changes made, number of tag statuses confirmed or modified, number of changes made to documentation, and so on.

FIGS. 2-9 show a user interface that is associated with the flowchart shown in FIG. 1. FIG. 2 shows an interface that allows a user to select an incident report to audit. In this example, a set of fall incidents are shown on screen. Each of these incident reports has been collected according to step 100. Information shown in this interface includes a date the incident occurred, a date the incident was last reviewed on, and a note identifying who is reviewing the incident report along with a review status (e.g., in progress, completed, and so on).

Once a report is selected (according to step 106), a user is shown documentation associated with the report, starting with details of the report as show in FIG. 3. In this portion of the interface, several tags have been recommended and are displayed per step 108, including “Nursing Description,” Resident Description,” and “Immediate Action Taken.” Each recommended tag has some associated documentation in the incident report, which is displayed in this interface as well. Tags are recommended based on the documentation that exists in the incident report. A user can make changes to incident report tags per step 110 and can add notes or comments per step 112.

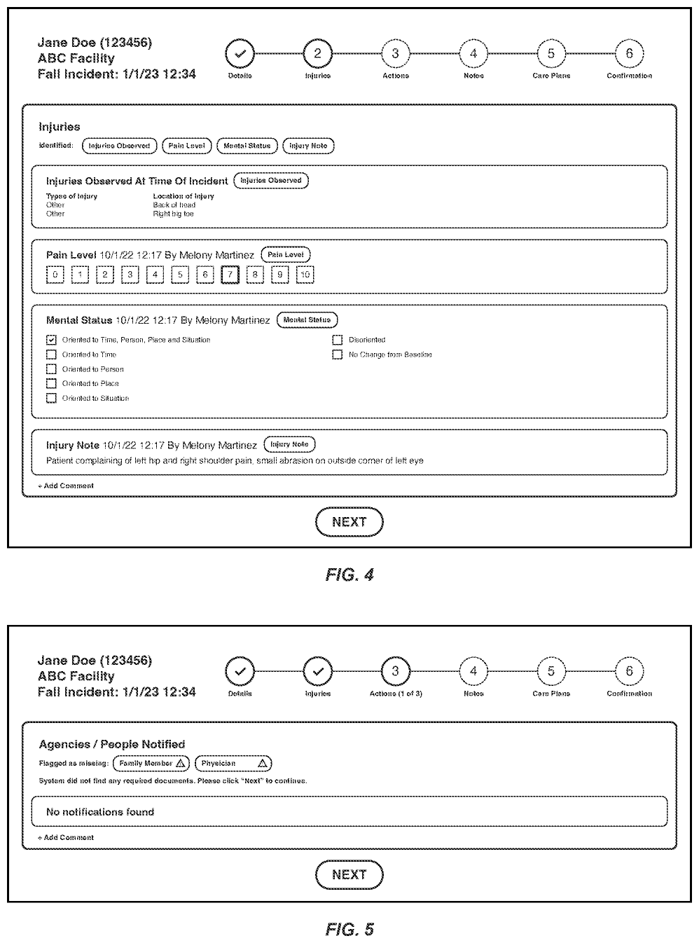

FIG. 4 shows the next user interface, which displays injuries associated with the incident along with some additional injury-related documentation. In this case, the incident report includes documentation that has been tagged with “Injuries Observed,” “Pain Level,” “Mental Status,” and “Injury Note.” Each tag corresponds to a portion of the incident report's documentation, and the corresponding documentation is shown in this same interface. In some situations, all the tags are correct, but in others, a user can change a tag to improve its descriptive accuracy. Additionally, documentation in an incident report can be reviewed and revised at this stage (or at any stage in which documentation is displayed to a user). The user can thus change tag statuses or edit tags according to step 110 and can add notes or comments per step 112.

In FIG. 5 that follows, missing documentation is flagged (e.g., per step 108). In this case, a fall incident may give rise to a need to notify family and a physician. The platform server has thus tagged documentation of those actions as missing (e.g., “Flagged as missing: ‘Family Member’ ‘Physician’). Tagging this documentation as missing allows a user to assess whether those actions were taken or need to be taken. If they were already taken, the user can add documentation to that effect by adding a new comment to the incident report, and if they were not taken, the user can ensure those actions are taken so the documentation can be updated later. The user can change tag statuses or edit tags according to step 110 and can add notes or comments per step 112.

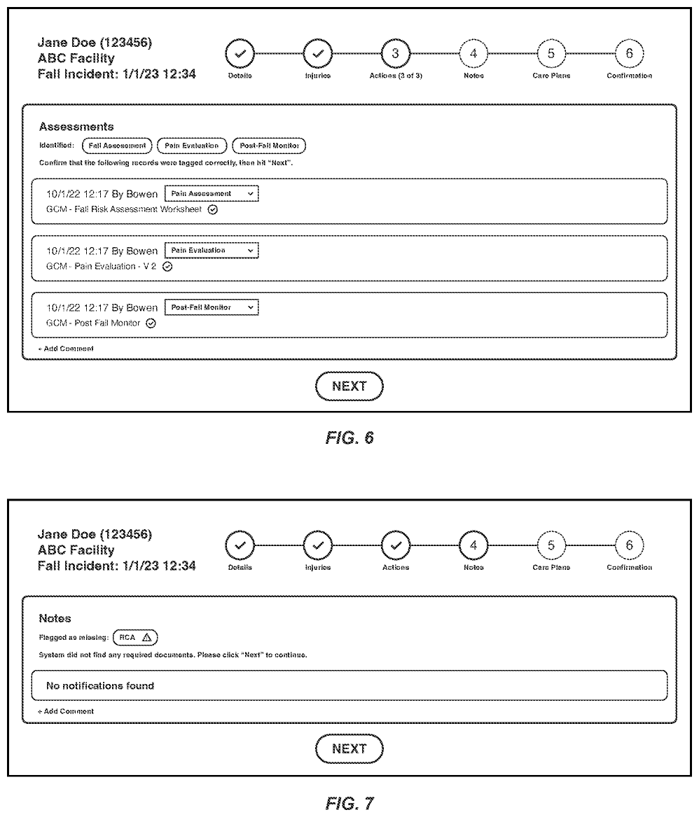

FIG. 6 shows actions that have been taken according to the incident report. In this case, the platform server has identified that a fall assessment and a pain evaluation along with post-fall monitoring has occurred. The user can verify the documentation of these actions by reviewing who entered them, at what time they were entered, the content of the documentation, and so on. The actions of the user on this interface correspond to step 110 where changes and edits can be made to documentation and documentation tags, and the user can also add or edit comments or notes per step 112.

FIG. 7 is an interface showing comments relating to an incident report. It shows that there is a missing root cause analysis (RCA) per step 108. This can prompt a user to add an RCA or to confirm that there is indeed no RCA. The user can change tag statuses or edit tags according to step 110 and can add notes or comments per step 112.

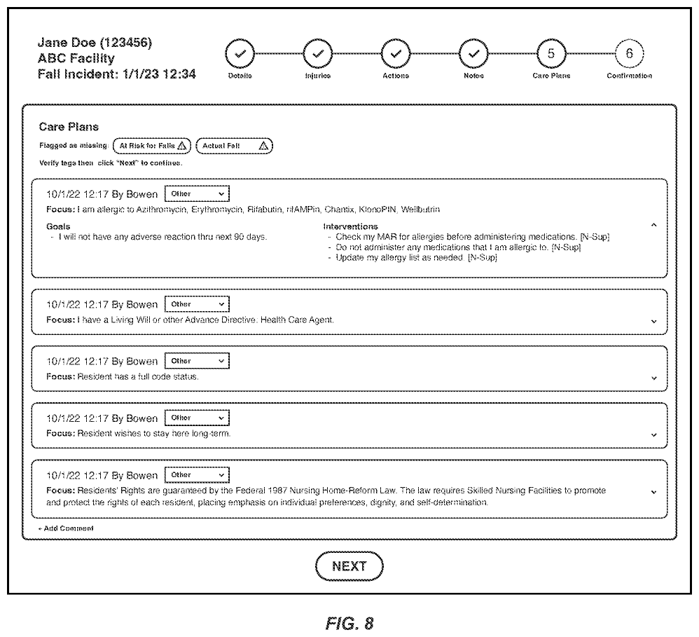

FIG. 8 is an interface showing care plans that are associated with an incident report. In this example, the platform server has identified that for this fall incident report, a care plan for a patient that is at risk of falls and a care plan for actual falls are both missing. This step allows a user to ensure best practices are implemented to care for patients going into the future. This interface also shows documentation that is relevant to care plans, including allergies to medications, notes on medical care directives, resident status, and resident wishes. This is a non-exhaustive list of documentation that can impact care plans.

Finally, FIG. 9 shows a user interface screen that a user would be presented with after auditing an incident report. This screen, per step 114, presents an opportunity for a user to assess whether any documentation or documentation tags are missing or need further revision before moving on to either steps 116 and 118 or to step 120. This interface shows information about the reported incident that was reviewed in the previous interfaces sorted into categories of risk management details.

Risk management details for the audited fall incident include, for example, categories for details, injuries, action, care planes, and notes. The details section includes a nurse description of the incident, a resident description of the incident, and an action taken. The injuries section includes a list of any injuries that occurred in association with the fall including a pain level, mental status, and an injury note, which all summarize information audited in previous interfaces. The action section includes recommended actions (e.g., actions tagged as missing) as well as actions that have already occurred. Here, the platform server recommends family notification and physician notification, both of which are missing, and it shows the existing progress note. The action section includes a listing of all assessments that have been undertaken, including a fall assessment, a pain evaluation, and post-fall monitoring, all with associated descriptions. The care plans section includes proposed care plans that can be implemented after a fall has occurred to help minimize future risks, including an “at risk for falls” and an “actual fall” care plan (each indicated as missing), and finally a notes section indicates the missing RCA.

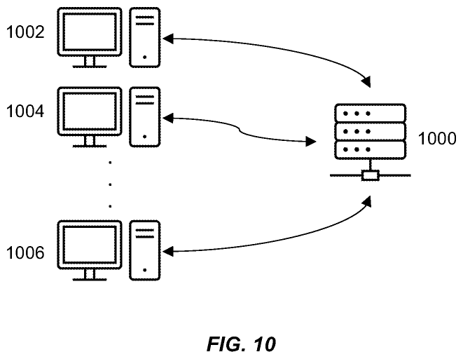

FIG. 10 is a schematic showing how computing devices used in systems and methods of the inventive subject matter can be connected. Platform server 1000 is shown having network connections (e.g., via the internet) to a series of computing devices 1002, 1004, and 1006. Although three computing devices are shown, the number of computing devices is restricted only by computing limitations that may restrict how many computers can connect to a server or set of servers. The platform server 1000 can be, e.g., a cloud server or other type of server that can be connected to via internet connection. Computing devices 1002, 1004, and 1006 represent computers that are used to access the platform service to use software as a service that exists on the platform server. That software as a service is what brings about the steps described in FIG. 1 as well as the user interfaces shown in FIGS. 2-9. Computing devices 1002, 1004, and 1006 can access the software as a service as either a user or an administration, depending on login credentials.

Systems and methods of the inventive subject matter can be used to achieve a variety of different goals, including: optimizing care facilities to receive Medicare and Medicaid reimbursements; developing risk management procedures in care facilities, to manage current medical, professional, and general liability medical claims; assisting in discovery requests required by lawsuits; assisting in underwriting insurance policies for care facilities and in adjusting prices of insurance premiums.

In some cases, the same audit processes disclosed in this application that are helpful in finding deficiencies in documentation can also be used to optimize reimbursement for Medicare and Medicaid billing. In some embodiments, the auditing process can be optimized for Medicare and Medicaid reimbursement based on requirements for such reimbursements.

Audit results can also sometimes be used to identify potential risks, such as problematic shifts or times of days where deficiencies are more likely to occur. A care facility can thus use an embodiment of the inventive subject matter to, e.g., recommend training to reduce the deficiencies during those time periods or with those staff members.

Embodiments can also enable export of relevant medical records to satisfy discovery requests related to incidents along with an audit trail showing that deficiencies were addressed. Embodiments can also allow a facility to establish that it has made improvements in completeness of documentation or improvements in a quantity of incident reports that can lead to insurance premium reductions by demonstrating decreased risk.

Although embodiments described above are directed to an example involving a nursing facility, systems and methods of the inventive subject matter can be implemented to any kind of facility that requires auditing on activities and actions that take place in that facility. This lends itself well to health care but can also be useful in the context of schools, workplaces, zoos, and so on.

Thus, specific systems and methods directed to human activity monitoring and auditing have been disclosed. It should be apparent, however, to those skilled in the art that many more modifications besides those already described are possible without departing from the inventive concepts in this application. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A method of auditing an incident report generated at a care facility, the method comprising the steps of:
receiving, by a platform server, the incident report from the care facility, the incident report comprising documentation of the incident;
applying, by the platform server, tags to the documentation, wherein each tag has an associated tag status;
receiving, by the platform server, an audit selection, the audit selection comprising the incident report;
displaying the documentation with the applied tags and tag statuses;
receiving, by the platform server, changes to the documentation, the applied tags, and the tag statuses; and
displaying a summary of all changes made to the documentation, the applied tags, and the tag statuses.

2. The method of claim 1, further comprising the step of identifying, by the platform server, the incident report as having been audited and storing a time of audit completion.

3. The method of claim 1, further comprising the step of receiving, after displaying the summary, an indication of whether additional documentation is missing.

4. The method of claim 3, wherein upon a determination that the additional documentation is missing, the platform server sends audit information to the care facility for additional review.

5. The method of claim 1, wherein each tag status comprises at least one of a “missing” status and an “incomplete” status.

6. A method of auditing incident reports generated at care facilities, the method comprising the steps of:
receiving, by a platform server, an incident report generated at a care facility, the incident report comprising documentation of an incident;
applying, by the platform server, a documentation tag to a portion of the documentation, wherein the documentation tag has a tag status;
storing, by the platform server, statistically useful information relating to the portion of the documentation based on the tag status;
receiving, by the platform server, an audit selection, the audit selection comprising the incident report;
displaying the documentation, including the portion of the documentation having the documentation tag and the tag status;
receiving, by the platform server, changes to documentation and tags; and
displaying a summary of changes made to documentation, including changes to the documentation tag and the tag status.

7. The method of claim 6, wherein the tag status comprises at least one of a “missing” status and an “incomplete” status.

8. The method of claim 6, further comprising the step of identifying, by the platform server, the incident report as having been audited and storing a time of audit completion.

9. The method of claim 6, further comprising the step of receiving, after displaying the summary, an indication of whether additional documentation is missing.

10. The method of claim 9, wherein upon a determination that the additional documentation is missing, the platform server sends audit information to the care facility for additional review.

11. The method of claim 6, wherein the statistically useful information is used in a statistical analysis.
